(19) United States
(12) Patent Application Publication
(10) Pub. No.: US 2023/0372613 A1
(43) Pub. Date: Nov. 23, 2023

(54) CUSTOMIZATION OF A GLUCOSE PREDICTION MODEL FOR A USER IN AN AUTOMATED INSULIN DELIVERY (AID) DEVICE

(71) Applicant: INSULET CORPORATION, Acton, MA (US)

(72) Inventors: Yibin ZHENG, Hartland, WI (US); Joon Bok LEE, Acton, MA (US); Jason O'CONNOR, Acton, MA (US)

(21) Appl. No.: 18/199,476

(22) Filed: May 19, 2023

Related U.S. Application Data
(60) Provisional application No. 63/343,739, filed on May 19, 2022.

(51) Int. Cl. A61M 5/172; A61M 5/142

(52) U.S. Cl. CPC A61M 5/1723; A61M 5/14244; A61M 2205/52

(57) ABSTRACT

The exemplary embodiments may employ a glucose prediction model (GPM) that is tailored to a user to account for insulin sensitivity or insulin insensitivity. The exemplary embodiments may predict future glucose levels based on past glucose levels for the user. Specifically, the GPM in exemplary embodiments may predict the future glucose level of the user as a weighted sum of most recent glucose level readings from the user. The exemplary embodiments may employ linear regression analysis to determine the values of the weights. These weights customize the GPM of the user based on the user's most recent glucose level history. Due to the customization, the GPM may more accurately predict future glucose levels of the user. As a result, the AID may exhibit better glucose level control for the user. The GPM of the exemplary embodiments may be updated on an ongoing basis.

### RELATED APPLICATIONS

This application claims the benefit of U.S. Provisional Patent Application No. 63/343,739, filed May 19, 2022, the entire contents of which are incorporated herein by reference in their entirety.

### BACKGROUND

An automated insulin delivery (AID) device delivers small amounts of insulin to a diabetic user to help regulate the glucose levels of the user. Typically, the small amounts of insulin are delivered at periodic intervals, such as at five-minute intervals. A control system in the AID device may determine the dosage of insulin to deliver at each interval. The control system may look at a number of different factors to determine the dosage of insulin to deliver. These factors may include a predicted future glucose level for the user. The control system may employ a glucose prediction model (GPM) that determines the predicted future glucose level for the user. The control system may compare the predicted future glucose level from the GPM with a target glucose level to determine a difference. The difference may dictate, in part, the insulin dosage delivered for the next or upcoming cycles. For example, if the GPM predicts that the glucose level of the user is going to be substantially above target, the control system may increase the insulin dosage delivered in the next cycle.

The GPM predicts the future glucose level well for many users. However, for users that are insulin sensitive or insulin insensitive, the GPM may not make very accurate predictions. This may be problematic in that the control system may be making decisions regarding insulin dosages based on inaccurate information. Moreover, there is an increased risk of poor glucose level control as a result of the inaccurate predicted future glucose levels that are predicted by the GPM.

### SUMMARY

In accordance with a first inventive facet, an insulin delivery device includes a reservoir for storing insulin and a non-transitory storage medium for storing computer programming instructions and past glucose levels for a user of the insulin delivery device. The insulin delivery device further includes a processor for executing the computer programming instructions to cause the processor to customize a glucose prediction model for the user for predicting future glucose levels of the user based on the past glucose level readings for the user and use the customized glucose prediction model in determining a basal insulin delivery dosage by the insulin delivery device. The computer programming instructions further cause the processor to cause the delivery of the determined basal insulin delivery dosage from the reservoir to the user.

The processor may be further configured to modify the glucose prediction model in view of more recent past glucose levels for the user and use the modified glucose prediction model in determining a next basal insulin delivery dosage by the insulin delivery device. The processor may be further configured to update the customizing of the glucose prediction model based on glucose levels received since the customizing, use the updated customized glucose prediction model in determining a new basal insulin delivery dosage by the insulin delivery device, and cause the insulin delivery device to deliver the determined new basal insulin delivery dosage. The customizing of the glucose prediction model may choose weight coefficient values used in the glucose prediction model. The customizing may entail using linear regression analysis to choose coefficient values that substantially minimize an error between predicted glucose levels that are predicted from past glucose levels for the user and corresponding actual glucose level readings for the user. The glucose prediction model may be linear. The glucose prediction model may ignore how much insulin has been delivered to the user.

In accordance with another inventive facet, a method is performed by a processor of an electronic device. The method includes determining values of weights for past glucose levels of a user of an insulin delivery device based on a glucose history for the user and applying the determined weights to the past glucose levels to produce weighted past glucose levels. The method also includes determining a predicted glucose level for a user at a given time as a sum of the weighted past glucose levels and using the predicted glucose level for the user to control delivery of insulin to the user by the insulin delivery device.

The determining of the values of the weights for the past glucose levels of the user of the insulin delivery device based on the glucose history for the user may entail, for selected glucose levels in the glucose history that includes glucose levels and associated times at which the glucose levels were sensed, calculating predicted glucose levels from weighted glucose levels in the glucose history for times that immediately precede the times of the selected glucose levels in the glucose history. The determining of the values of the weights may entail performing least squares regression analysis with the past glucose levels and predicted glucose levels that are predicted from the past glucose levels. A predicted glucose level may be calculated as a sum of the weighted glucose levels in the glucose history for times that immediately precede a time of the given one of the predicted glucose levels. The method may further include comparing the predicted glucose level to a high glucose level threshold and where the predicted glucose level exceeds the high glucose level threshold, taking corrective action. The corrective action may include one or more of outputting an alert, outputting a recommendation or delivering an insulin bolus to the user. The method may further include comparing the predicted glucose level to a low glucose level threshold and where the predicted glucose level falls below the low glucose level threshold, taking corrective action. The corrective action may include one or more of outputting an alert, outputting a recommendation to ingest rescue carbohydrates or delivering a glucagon bolus to the user.

In accordance with a further inventive facet, an electronic device includes a storage for storing computer programming instructions for controlling operation of a insulin delivery device and a processor for executing the computer programming instructions. The computer programming instructions are for causing the processor to use a glucose prediction model to predict future glucose levels of a user of the insulin delivery device and to customize the glucose prediction model of the user based on past glucose levels of the user. The computer programming instructions also are for causing the processor to use the customized glucose prediction model to predict future glucose levels of the user and use at least one of the predicted future glucose levels in determining a basal delivery dosage of insulin to be delivered to the user from the insulin delivery device.

The electronic device may be the insulin delivery device or a management device of the insulin delivery device. The computer programming instructions may include instructions for causing the processor to update the customizing of the glucose prediction model based on more recent glucose levels for the user. The computer programming instructions may include instructions for causing the processor to adjust the predicted glucose levels for the user to account for noise. The glucose prediction model may not account for insulin delivered to the user in predicting the future glucose levels of the user.

### BRIEF DESCRIPTION OF THE DRAWINGS

FIG. 1 depicts an illustrative medicament delivery system suitable for exemplary embodiments.

FIG. 2 depicts a flowchart of illustrative steps that may be performed in exemplary embodiments to predict a future glucose level of a user.

FIG. 3 depicts a flowchart of illustrative steps that may be performed in exemplary embodiments to customize a glucose prediction model (GPM) of the user.

FIG. 4 depicts an example of illustrative matrices that may be used in exemplary embodiments to customize the GPM of the user.

FIG. 5 depicts a flowchart of illustrative steps that may be performed in exemplary embodiments to determine weights for the GPM.

FIG. 6 depicts a flowchart of illustrative steps that may be performed in exemplary embodiments to update the GPM of the user.

FIG. 7 depicts illustrative triggers for updating the GPM customization for the user.

FIG. 8A depicts a flowchart of illustrative steps that may be performed in exemplary embodiments to respond to a high glucose level for a predicted future glucose level of the user.

FIG. 8B depicts illustrative corrective actions that may be taken responsive to determining that the predicted future glucose level of the user exceed a high glucose threshold in exemplary embodiments.

FIG. 9A depicts a flowchart of illustrative steps that may be performed in exemplary embodiments to respond to a low glucose level of a predicted future glucose level of the user.

FIG. 9B depicts illustrative corrective actions that may be taken responsive to determining that the predicted future glucose level of the user falls below a low glucose threshold in exemplary embodiments.

FIG. 10 depicts a flowchart of illustrative steps that may be performed in exemplary embodiments to adjust a basal delivery dosage responsive to a predicted future glucose level of the user.

FIG. 11 depicts a flowchart of illustrative steps that may be performed in exemplary embodiments to compensate for possible noise in glucose level readings from a sensor.

### DETAILED DESCRIPTION

The exemplary embodiments may employ a GPM that is tailored to a user to account for insulin sensitivity or insulin insensitivity. The Exemplary embodiments provide an approach to adapt the parameters of a GPM in real time based on least squares regression with regularization of glucose predictions versus actual glucose values. The exemplary embodiments may predict future glucose levels based on past glucose levels of the user. Specifically, the GPM in exemplary embodiments may predict the future glucose level of the user as a weighted sum of most recent glucose levels from the user (such as the most recent glucose level readings from a glucose monitor). The exemplary embodiments may employ linear regression analysis to determine the values of the weights. These weights customize the GPM of the user based on the user's most recent glucose level history. Due to the customization, the GPM may more accurately predict future glucose levels of the user. As a result, the AID may exhibit better glucose level control for the user.

The GPM of the exemplary embodiments may be updated on an ongoing basis. As new glucose level readings arrive, the weights may be updated to reflect the more recent glucose level readings for the user.

The GPM may also be updated so as to minimize the effect of noise. The exemplary embodiments may limit the amount of change in weights between updates so as to avoid more significant changes that may be the result of noisy glucose level readings for the user. This approach changes more slowly as a result but avoids the complication of noise.

The GPM is a model for predicting the future glucose levels of the user. The GPM need not be a formalized model but rather refers to a strategy for predicting the future glucose levels. The GPM may be a simple linear equation or even a heuristic in some instances. The approach adopted by the GPM may be non-linear in alternative embodiments.

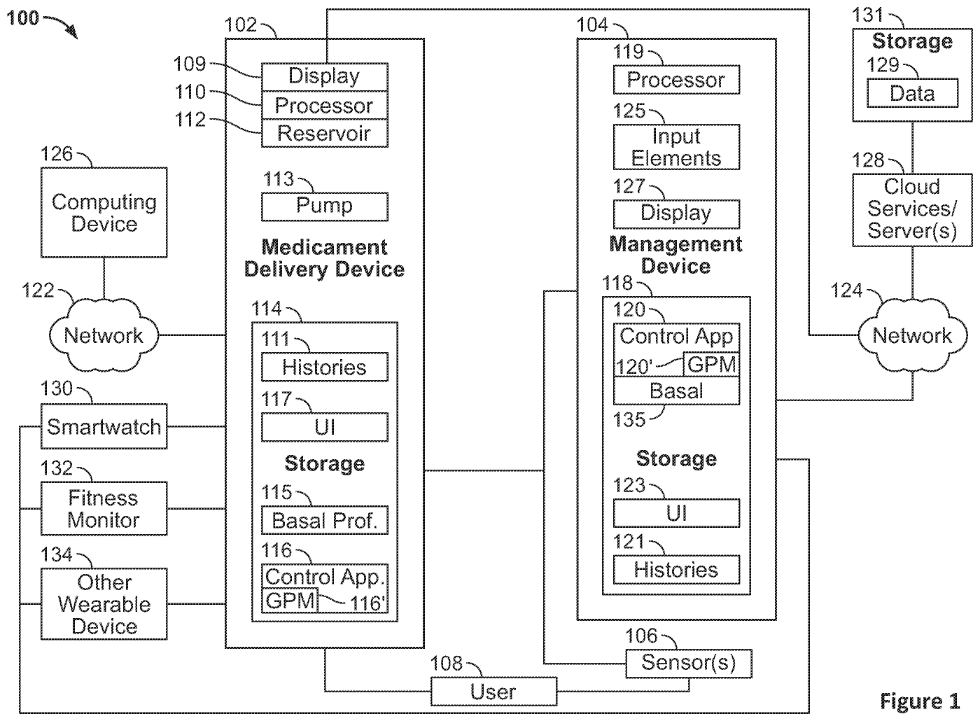

FIG. 1 depicts an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user 108. The medicament delivery device 102 may be directly coupled to the user 108 (e.g., directly attached to a body part and/or skin of the user 108 via an adhesive or the like) or carried by the user 108 (e.g., on a belt or in a pocket) with the medicament delivery device 102 being connected to an infusion site where the medicament is injected using a needle and/or cannula. In a preferred embodiment, a surface of the medicament delivery device 102 may include an adhesive to facilitate attachment to the user 108. For purposes of the discussion below it is presumed that the medicament delivery device 102 is an insulin delivery device that delivers insulin. The medicament delivery device may also deliver other medicaments, such as glucagon, GLP-1, pramlintide, or co-formulations of medicaments.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be realized as a single program, multiple programs, modules, libraries, or the like. The control application 116 may be responsible for implementing the control system that provides feedback and adjustments to medicament dosages that are delivered to the user 108. The control application 116, in some exemplary embodiments, may implement the GPM 116′ and provide the functionality detailed below. The processor 110 also may execute computer programming instructions stored in the storage 114 for a user interface 117 that may include one or more display screens shown on display 109. The display 109 may display information to the user 108 and, in some instances, may receive input from the user 108, such as when the display 109 is a touchscreen.

The control application 116 may control delivery of a medicament to the user 108 per a control approach like that described herein. The storage 114 may hold histories 111 for the user 108, such as a history of basal deliveries, a history of bolus deliveries, and/or other histories, such as a meal event history, exercise event history, glucose level history, medicament delivery history, sensor data history and/or the like. These histories 111 may be processed as will be described below to adjust basal medicament dosages to help reduce or eliminate persistent positive low level medicament excursions. The storage 114 also may include one or more basal profiles 115 that are used when the medicament delivery device is operating in open loop mode. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include one or more housings for housing its various components including a pump 113, a power source (not shown), and a reservoir 112 for storing a medicament for delivery to the user 108. The medicament in the reservoir 112 may be insulin, for example, or the other medicaments noted above. In some embodiments, the reservoir may be partitioned to store another medicament as well, such as glucagon, or one of the other medicaments noted above. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112) and may include a conduit to a separate infusion site.

There may be one or more communications links with one or more devices physically separated from the medicament delivery device 102 including, for example, a management device 104 of the user 108 and/or a caregiver of the user 108, a sensor 106, a smartwatch 130, a fitness monitor 132 and/or another variety of wearable device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network, and the computing device may communicate with the medicament delivery device 102 or the management device 104.

The medicament delivery system 100 may include one or more sensor(s) 106 for sensing the levels of one or more analytes or for sensing environmental conditions. Examples of sensors 106 include a continuous glucose monitor (CGM), a hear rate monitor, a blood pressure monitor, a temperature sensor, a barometer, an accelerometer, etc. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof.

The medicament delivery system 100 may or may not also include management device 104. In some embodiments, a management device is not needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensors 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The control application 120 may be responsible for controlling the medicament delivery device 102, such as by controlling the AID delivery of insulin to the user 108. The control application 120 may implement the GPM 120′ in some embodiments. The control application 120 may customize the GPM 120′ and implement the functionality described below. The storage 118 may store the control application 120, histories 121 like those described above for the medicament delivery device 102, one or more basal profiles 135 and other data and/or programs.

A display 127, such as a touchscreen, may be provided for displaying information. The display 127 may display user interface (UI) 123. The display 127 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks via wired or wireless communication links. The management device 104 may communicate over network 124 with one or more servers or cloud services 128. Data, such as sensor values like glucose levels, may be sent, in some embodiments, for storage and processing from the medicament delivery device 102 directly to the cloud services/server(s) 128 or instead from the management device 104 to the cloud services/server(s) 128. The cloud services/server(s) 128 may provide output from the model 115 as needed to the management device 104 and/or medicament delivery device 102 during operation.

Other devices, like smartwatch 130, fitness monitor 132 and wearable device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user 108, such as to request a change or pause in dosage or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

The functionality described below for the exemplary embodiments may be under the control of or performed by the control application 116 of the medicament delivery device 102 or the control application 120 of the management device 104. In some embodiments, the functionality may be under the control of or performed by the cloud services or servers 128, the computing device 126 or by the other enumerated devices, including smartwatch 130, fitness monitor 132 or another wearable device 134.

The medicament delivery device 102 may operate in an open loop mode and in a closed loop mode. In the open loop mode, the user 108 manually inputs the amount of medicament to be delivered (such as per hour) for segments of the day. The inputs may be stored in a basal profile 115, 135 for the user 108. In other embodiments, a basal profile may not be used. The control application 116, 120 uses the input information from the basal profile 115, 135 to control basal medicament deliveries in open loop mode. In contrast, in the closed loop mode, the control application 116, 120 determines the medicant delivery amount for the user 108 on an ongoing basis based on a feedback loop. For an insulin delivery device, the aim of the closed loop mode is to have the user's glucose level at a target glucose level or within a range of glucose levels. The basal dosages may be delivered at fixed regular intervals, designated as cycles, such as every five minutes, though may vary in amount for each cycle. The GPM 116′ or 120′ is used in closed loop mode.

In the exemplary embodiments, the functionality described below may be realized by executing the control application 116 or 120 or by running a control application on other devices, such as smartwatch 130, fitness monitor 132 or other type of wearable device 134. More generally, the functionality may be realized by computer programming instructions executing on a processor for controlling the medicament delivery device 102.

The customization and use of the GPM described below may be performed by control application 116 of the medicament delivery device 102 (i.e., the AID device) or by the control application 120 of the management device 104. Some functionality and/or operations may be performed by the smartwatch 130, the fitness monitor 132, the other wearable device 134, the computing device 126 and/or the cloud services/server 128 in some embodiments.

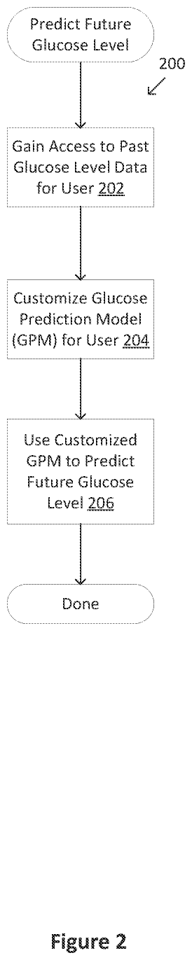

The exemplary embodiments may more accurately predict future glucose levels for a user 108 than conventional AID devices. FIG. 2 depicts a flowchart 200 of illustrative steps that may be performed in exemplary embodiments to predict future glucose levels for a user 108. Initially, at 202, access to past glucose level data for the user. The past glucose level data, for example, may be stored in storage 114 of the medicament delivery device 102 as part of the histories 111 or may be stored in the storage 118 of the management device 104 as part of the histories 121. The past glucose level data may even be obtained in some embodiments from the sensor(s) 106, such as from a glucose monitor, like a CGM. The past glucose level data may be processed as described in more detail below to customize the GPM 116′ or 120′ for the user 108 at 206. The customized GPM 116′ or 120′ may then be used to predict at least one and likely multiple future glucose levels for the user 108 at 206.

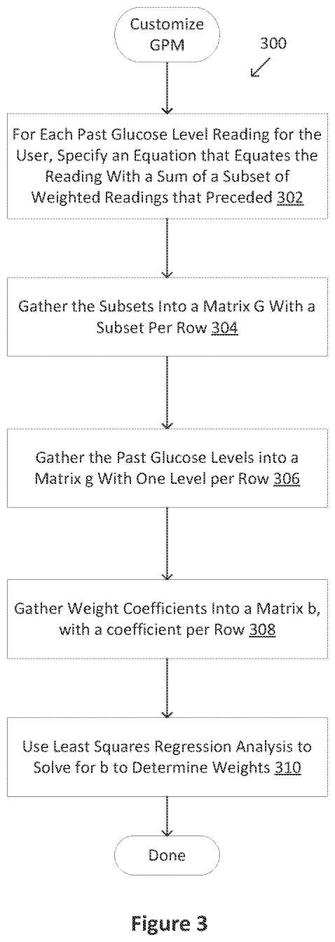

FIG. 3 depicts a flowchart of illustrative steps that may be performed in exemplary embodiments to customizer the GPM 116′ or 120′. For each past glucose level in a subset that is to be used to determine weights, at 302, an equation is specified that equates the glucose level with the weighted some of the previous glucose levels that preceded. For example, for a glucose level value G(k) for cycle k, the equation may be expressed as:

G(k)=b*₁G(k−1)+b*₂G(k−2)+b*₃G(k−3)

where b* is a weight, also referred to as a weight coefficient, where b*₁, b*₂, and b*₃ represent different weight coefficients for different cycles. A cycle refers to an operational cycle of the medicament delivery device 102. Each cycle may last a fixed period of time, such as 5 minutes, and a basal delivery dosage may be determined for each cycle. Although this example assesses the value of current glucose concentration across three previous cycles (applying an equation for up to G(k−3)), this formulation can be extended into a greater number of previous cycles, each additional cycle having its own weight b* such as b*₄, b*₅ . . . etc.

For the previous cycle k−1, the equation is:

G(k−1)=b*₁G(k−2)+b*₂G(k−3)+b*₃G(k−4).

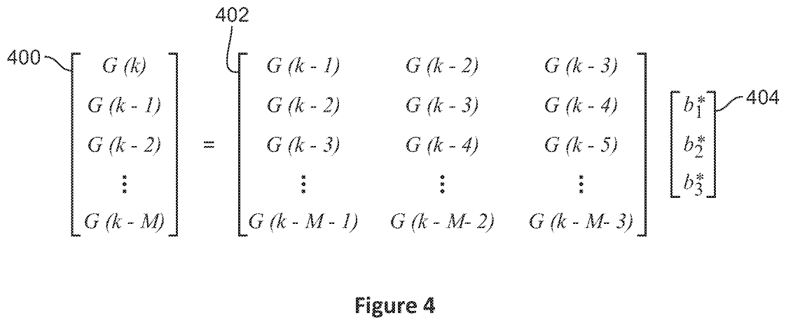

M+1 of these equations may be used in the customization. M is an integer and is a customizable value. At 304, the subset of previous glucose levels that are used in predicting an associated glucose level are gathered into a matrix G with one subset per row. As above, these equations can also be extended further to additional previous cycles beyond G(k−4). FIG. 4 depicts an example 402 of the matrix G. At 306, the past glucose levels being predicted by the subsets are gathered into a matrix g with one past glucose level per row. FIG. 4 depicts an example 400 of the matrix g. At 308, the weight coefficients are gathered into a matrix b with one weight per row. FIG. 4, depicts an example 404 of the matrix b. At 310, least squares regression analysis may be applied to determine the weights. Least squares regression approximates a solution of overdetermined systems by minimizing the sum of the residuals, where a residual is a difference between and observed value and a fitted value provided by a model. The least squares regression analysis chooses weights that minimize the error between the actual glucose levels and the predictions from the past glucose levels. It should be appreciated that other linear regression techniques may be used or even non-linear techniques may be used. The size of the matrix G is M by 3, where M is the length of glucose historical data used to calculate the least squares weights b*₁ b*₂ b*₃ and 3 is the prediction model order. For example, one day's data may be used to fit a 3^{rd} order least squares model. In this case the matrix G will be 288 by 3, where 288 represents the number of 5-minutes cycles in one day.

As shown in FIG. 4, the matrix g is the product of matrix multiplication of the matrices G and b. This can be expressed as g=Gb, where the dimensions of g, G, and b are M by 1, M by 3, and 3 by 1 respectively. This formulation ignores insulin delivered to the user 108. The insulin contributes little to the predicted glucose level relative to the past glucose levels so the insulin delivered to the user 108 may be discounted and not part of the calculation for the predicted future glucose level. To solve for b, both sides of the equation may be multiplied by the transpose of G, designated as G^{T} to yield G^{T}g=G^{T}G b. Then, one can solve for b by multiplying by the inverse of G^{T}G. The result is b=(G^{T}G)⁻¹G^{T}g.

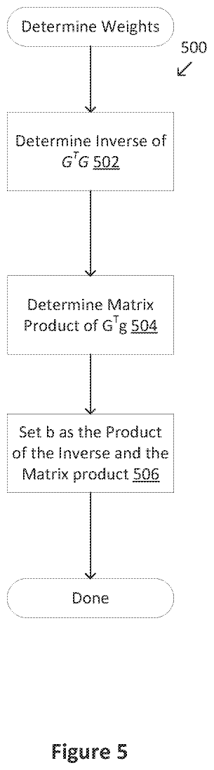

Given this formulation for b, FIG. 5 depicts a flowchart 500 of illustrative steps that may be performed in exemplary embodiments to determine the weights matrix b and as a result, customize the GPM 116′ or 120′. At 502, the inverse of G^{T}G is calculated. At 504, the matrix product of G^{T}g is determined. At 506, the matrix b is set as the product of the inverse of 502 and the matrix product of 506.

Since in the example G is a M×3 matrix, G^{T}G results in a 3×3 matrix, as is the inverse. When the inverse is multiplied by G^{T}, a 3×M matrix, the result is a 3×M matrix. When this matrix is multiplied by the matrix g, which is a M×1 matrix, the result is a 3×1 matrix for b. Note that the sizes of these matrices can be varied based on the model order of prediction. Specifically, if the duration of previous cycles is increased from 3 to N, the example G matrix may be an M×N matrix, and the result of the multiplication with G^{T} will be an N×N matrix. The final outcome will be an N×1 matrix for b, corresponding to the weights of historic glucose data samples.

The least squares weights b may be recalculated periodically, e.g., every 6 or 24 hours, they may be recalculated when triggered by certain events as described below, or they may be continuously updated per each cycle of operation.

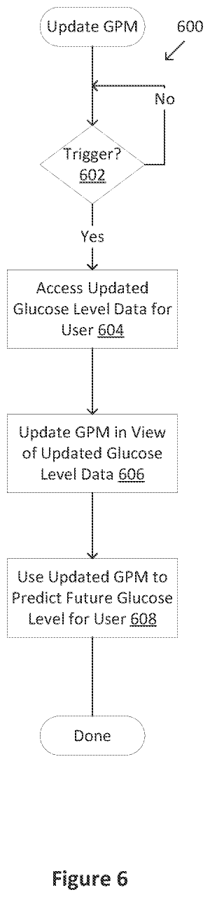

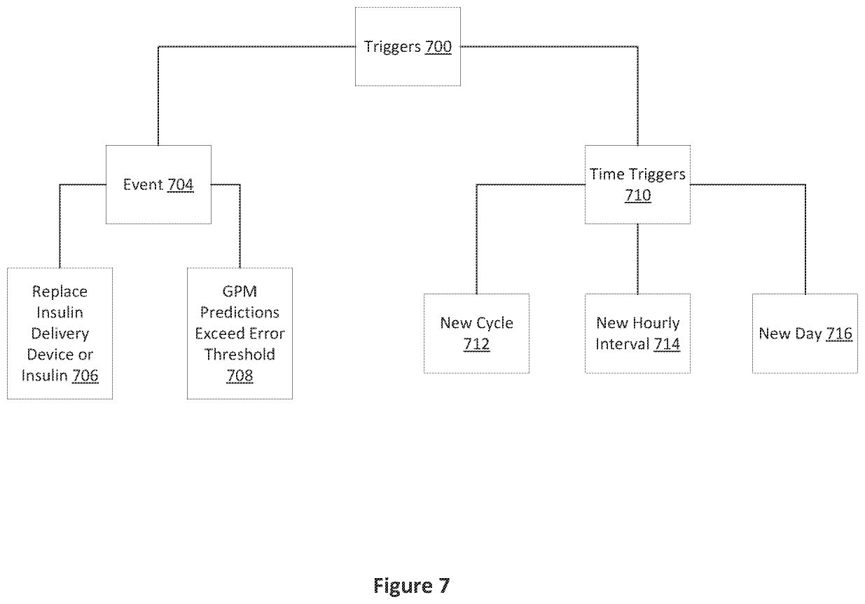

As was mentioned above, the GPM 116′ or 120′ may be updated to reflect more recent glucose level data for the user 108. FIG. 6 depicts a flowchart 600 of illustrative steps that may be performed to update the customization of the GPM 116′ or 120′ by updating the weights. At 602, a check is made whether a trigger has been reached. As shown in FIG. 7, a number of different types of triggers 700 may be used. In some embodiments, an update may be triggered by an event 704. Examples of events that may be triggering are the replacement of the insulin delivery device or insulin supply 706. Some insulin delivery devices are designed to be worn a fixed period of time, such as three days, and then replaced. Similarly, an insulin supply may be replaced after a fixed period of time, such as every few days. These events 706 may trigger an updating of the customization of the GPM 116′ or 120′. Another example of an event that may trigger an update is if the GPM predictions of future glucose levels exceed a tolerance threshold 708. Other events may also trigger an update.

The trigger instead may be a time based trigger 710. For example, a new cycle beginning 712 (i.e., every 5 minutes) may trigger an update to the customization of the GPM 116′ or 120′. A new hourly interval 714 may trigger an update. For instance, an update may occur every hour, every 3 hours or every 12 hours. A new day 716 may trigger an update. It should be appreciated that other time intervals may be used to trigger the updates.

If triggered at 602, at 604, updated glucose level data is accessed. For instance several new glucose level readings may have been received from the sensor(s) 106. At 606, the GPM 116′ or 120′ is updated in response to the trigger to account the new glucose level readings. At 608, the updated GPM 116′ or 120′ is used to predict at least one future glucose level for the user 108.

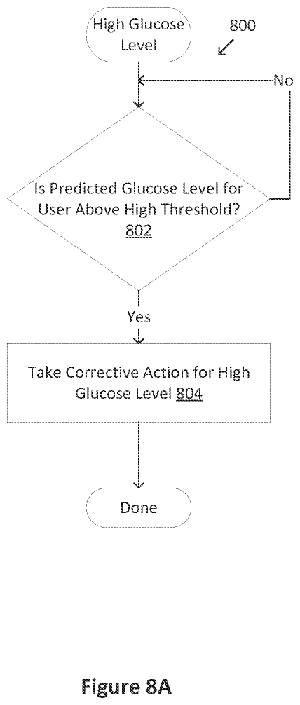

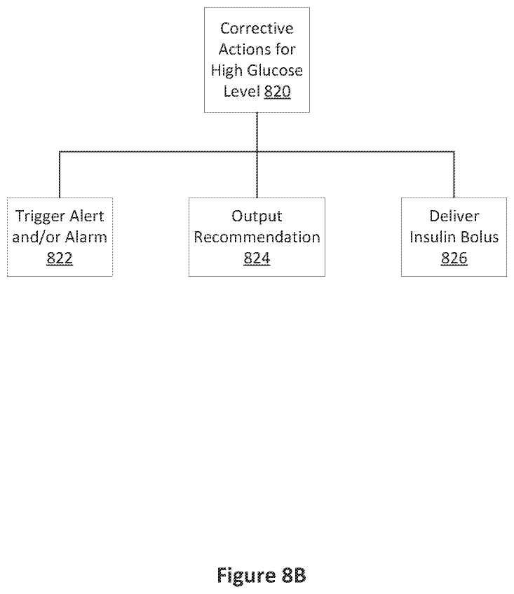

The predicted glucose level for the user from the GPM 116′ or 120′ may be used in a number of different ways. FIG. 8A depicts a flowchart 800 of illustrative steps that may be performed with respect to a high glucose level. At 802, a check is made whether the predicted glucose level exceeds a high threshold, such as a hyperglycemic threshold or another high threshold. If so, corrective action may be taken at 804. FIG. 8B depicts examples of corrective actions 820 for a high glucose level. One or more of these corrective actions 820 may be taken. An alert or alarm may be triggered 822 to alert the user 108. The alert may be a graphic or a textual message displayed on display 109 and/or display 127. The alarm may be visual and/or auditory. A recommendation may be output 824, such as on display 109 and/or display 127. For example, the recommendation may be a message to exercise or deliver an insulin bolus to reduce the glucose level of the user 108. Another corrective action is to deliver an insulin bolus 826 to the user 108 via the medicament delivery device 102.

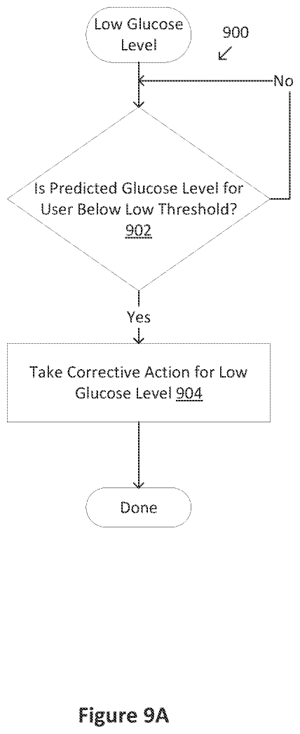

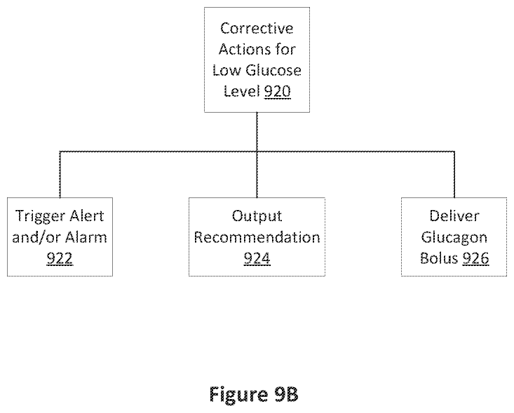

FIG. 9A depicts a flowchart 900 of illustrative steps that may be performed with respect to a low glucose level. At 802, a check is made whether the predicted glucose level falls below a low threshold, such as a hypoglycemic threshold or another low threshold. If so, corrective action may be taken at 904. FIG. 9B depicts examples of corrective actions for a low glucose level. One or more of these corrective actions 920 may be taken. An alert or alarm may be triggered 922 to alert the user 108. The alert may be a graphic or a textual message shown or display 109 and/or display 127. The alarm may be visual or auditory. A recommendation may be output 924 on display 109 and/or display 127. For example, the recommendation may be a message to ingest rescue carbohydrates to raise the glucose level of the user 108. Another corrective action is to deliver a glucagon bolus 926 to the user 108 via the medicament delivery device 102 or another medicament delivery device such as a medicament pen device.

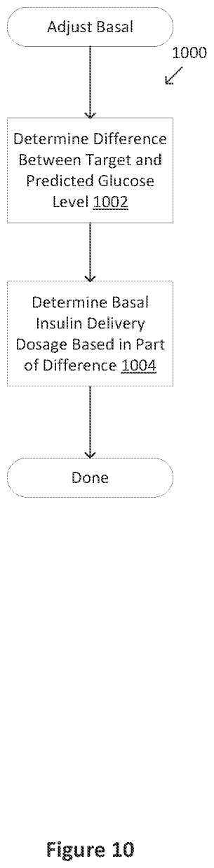

Another action that may result from the customization of the GPM 116′ or 120′ is an adjustment is the basal delivery dosage from the medicament delivery device 102. FIG. 10 depicts a flowchart 1000 of steps that may be performed to update the basal dosage by the control system of the medicament delivery device 102. The updated GPM 116′ or 120′ generates the predicted glucose level and the difference between the predicted glucose level and a target glucose level is determined at 1002. Based on this difference, the basal dosage for at least the next basal delivery is updated at 1004 by the control system (e.g., control application 116).

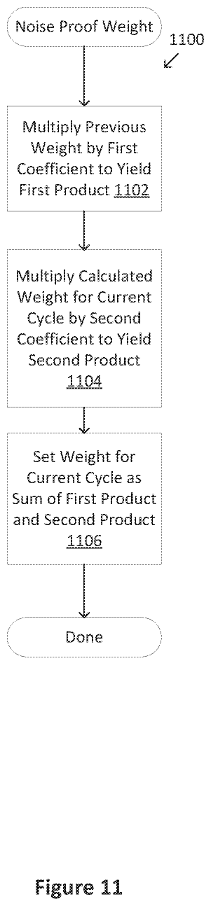

One problem that may arise with the customization of the GPM 116′ or 120′ is that one or more noisy glucose level reading from sensor 106 may have an undue effect on the weights in matrix b. Hence, steps may be taken to reduce the effects of the noise by only making incremental changes so that the effects of noise are minimized. FIG. 11 depicts a flowchart 1100 of illustrative steps that may be performed to offset the effects of noise. At 1102, the previous weight value is multiplied by a weight coefficient to yield a first product. The weight coefficient should be a large weight, such as 0.9, to prevent the weight from changing dramatically. At 1104, the calculated updated weight (i.e., the weight calculated as the result of the update as described above) is multiplied by a second weight coefficient to yield a second product. A suitable value for the second weight coefficient is 0.1. Both of these weight coefficients must range between 1 and 0, and the sum of these weight coefficients must always equal 1. At 1106, the weight for the current cycle is calculated as the sum of the first product and the second product. For example, the weight b_{final} may be calculated as:

B_{final}=0.9b_{final}(k−1)+0.1b*(k−1).

The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device 102, e.g. the storage 114, or on the management device 104, e.g. the storage 118.

While exemplary embodiments have been described herein, it should be appreciated that various in form and detail may be made without departing from the intended scope as defined in the appended claims.

1. An insulin delivery device, comprising:
a reservoir for storing insulin;
a non-transitory storage medium for storing computer programming instructions and past glucose levels of a user of the insulin delivery device;
a processor for executing the computer programming instructions to cause the processor to:
customize a glucose prediction model of the user for predicting future glucose levels of the user based on the past glucose level readings of the user;
use the customized glucose prediction model in determining a basal insulin delivery dosage by the insulin delivery device; and
cause the delivery of the determined basal insulin delivery dosage from the reservoir to the user.

2. The insulin delivery device of claim 1, wherein the processor is further configured to modify the glucose prediction model in view of more recent past glucose levels of the user and use the modified glucose prediction model in determining a next basal insulin delivery dosage by the insulin delivery device

3. The insulin delivery device of claim 1, wherein the processor is further configured to:
update the customizing of the glucose prediction model based on glucose levels received since the customizing;
use the updated customized glucose prediction model in determining a new basal insulin delivery dosage by the insulin delivery device; and
cause the insulin delivery device to deliver the determined new basal insulin delivery dosage.

4. The insulin delivery device of claim 1, wherein the customizing of the glucose prediction model comprises calculating weight coefficient values used in the glucose prediction model.

5. The insulin delivery device of claim 4, wherein the customizing entails using linear regression analysis to calculate coefficient values that substantially minimize an error between predicted glucose levels that are predicted from past glucose levels of the user and corresponding actual glucose level readings of the user.

6. The insulin delivery device of claim 1, wherein the glucose prediction model is linear.

7. The insulin delivery device of claim 1, wherein the glucose prediction model ignores how much insulin has been delivered to the user.

8. A method performed by a processor of an electronic device, comprising:
determining values of weights for past glucose levels of a user of an insulin delivery device based on a glucose history of the user;
applying the determined weights to the past glucose levels to produce weighted past glucose levels;
determining a predicted glucose level for a user at a given time as a sum of the weighted past glucose levels; and
using the predicted glucose level of the user to control delivery of insulin to the user by the insulin delivery device.

9. The method of claim 8, wherein the determining the values of the weights for the past glucose levels of the user of the insulin delivery device based on the glucose history of the user comprises:
for selected ones of the glucose levels in the glucose history that includes glucose levels and associated times at which the glucose levels were sensed, calculating predicted glucose levels from weighted glucose levels in the glucose history for times that immediately precede the times of the selected ones of the glucose levels in the glucose history.

10. The method of claim 9, wherein the determining of the values of the weights entails performing least squares regression analysis with the past glucose levels and predicted glucose levels that are predicted from the past glucose levels.

11. The method if claim 10, wherein a given one of the predicted glucose levels is calculated as a sum of the weighted glucose levels in the glucose history for times that immediately precede a time of the given one of the predicted glucose levels.

12. The method of claim 8, further comprising:
comparing the predicted glucose level to a high glucose level threshold; and
where the predicted glucose level exceeds the high glucose level threshold, taking corrective action.

13. The method of claim 12, wherein the corrective action comprises one or more of outputting an alert, outputting a recommendation or delivering an insulin bolus to the user.

14. The method of claim 8, further comprising:
comparing the predicted glucose level to a low glucose level threshold; and
where the predicted glucose level falls below the low glucose level threshold, taking corrective action.

15. The method of claim 14, wherein the corrective action comprises one or more of outputting an alert, outputting a recommendation to ingest rescue carbohydrates or delivering a glucagon bolus to the user.

16. An electronic device, comprising:
a storage for storing computer programming instructions for controlling operation of an insulin delivery device;
a processor for executing the computer programming instructions, the computer programming instruction for causing the processor to:
use a glucose prediction model to predict future glucose levels of a user of the insulin delivery device;
customize the glucose prediction model of the user based on past glucose levels of the user;
use the customized glucose prediction model to predicts future glucose levels of the user; and
use at least one of the predicted future glucose levels in determining a basal delivery dosage of insulin to be delivered to the user from the insulin delivery device.

17. The electronic device of claim 16, wherein the electronic device is one of the insulin delivery device or a management device for the insulin delivery device.

18. The electronic device of claim 16, wherein the computer programming instructions include instructions for causing the processor to update the customizing of the glucose prediction model based on more recent glucose levels of the user.

19. The electronic device of claim 16, wherein the computer programming instructions include instructions for causing the processor to adjust the predicted glucose levels of the user to account for noise.

20. The electronic device of claim 16, wherein the glucose prediction model does not account for insulin delivered to the user in predicting the future glucose levels of the user.
